SEQUENCE LISTING (16 sequences)

SEQ ID NO: 1 - DNA, 66 bp, Artificial Sequence
  The pH responsive tetrahedra
acactacgtcagaacagcttgcatcactggtcaccagagtaccctaaccctaaccctaaccctact

SEQ ID NO: 2 - DNA, 41 bp, Artificial Sequence
  The pH responsive tetrahedra
ggtcctacgagcgagttgatgtgatgcaagctgaatgcgag

SEQ ID NO: 3 - DNA, 70 bp, Artificial Sequence
  The pH responsive tetrahedra
ggtggtggtggttgtggtggtggtggttttctgacgtagtgtatgcacagtgtagttaggaccctcgcat

SEQ ID NO: 4 - DNA, 41 bp, Artificial Sequence
  The pH responsive tetrahedra
tcaactcgctcgttactacactgtgcaatactctggtgacc

SEQ ID NO: 5 - DNA, 12 bp, Artificial Sequence
  The pH responsive tetrahedra
  misc_feature: NH2-(CH2)6
agtagggttagg

SEQ ID NO: 6 - DNA, 51 bp, Artificial Sequence
  The pH responsive tetrahedra
ggtggtggtggttgtggtggtggtggccctaaccctaaccctaaccctact

SEQ ID NO: 7 - DNA, 66 bp, Artificial Sequence
  The miRNA-21 responsive tetrahedra (TA)
acactacgtcagaacagcttgcatcactggtcaccagagtaaaaaaaacatcagtctgataagcta

SEQ ID NO: 8 - DNA, 71 bp, Artificial Sequence
  The miRNA-21 responsive tetrahedra (TA)
acactacgtcagaacagcttgcatcactggtcaccagagtaaaatcaacatcagtctgataagctaaaaaa

SEQ ID NO: 9 - DNA, 41 bp, Artificial Sequence; identical to SEQ ID NO: 2
  The miRNA-21 responsive tetrahedra (TA)

SEQ ID NO: 10 - DNA, 41 bp, Artificial Sequence
  The miRNA-21 responsive tetrahedra (TA)
tctgacgtagtgtatgcacagtgtagttaggaccctcgcat

SEQ ID NO: 11 - DNA, 41 bp, Artificial Sequence; identical to SEQ ID NO: 4
  The miRNA-21 responsive tetrahedra (TA)

SEQ ID NO: 12 - DNA, 67 bp, Artificial Sequence
  The miRNA-155 responsive tetrahedra (TB)
acactacgtcagaacagcttgcatcactggtcaccagagtaaaaaaacctatcacgattagcattaa

SEQ ID NO: 13 - DNA, 71 bp, Artificial Sequence; identical to SEQ ID NO: 8
  The miRNA-155 responsive tetrahedra (TB)

SEQ ID NO: 14 - DNA, 41 bp, Artificial Sequence; identical to SEQ ID NO: 2
  The miRNA-155 responsive tetrahedra (TB)

SEQ ID NO: 15 - DNA, 41 bp, Artificial Sequence; identical to SEQ ID NO: 10
  The miRNA-155 responsive tetrahedra (TB)

SEQ ID NO: 16 - DNA, 41 bp, Artificial Sequence; identical to SEQ ID NO: 4
  The miRNA-155 responsive tetrahedra (TB)

(19) United States
(12) Patent Application Publication
(10) Pub. No.: US 2024/0082169 A1
(43) Pub. Date: Mar. 14, 2024

(54) NANOPARTICLE CARRIERS OF ACTIVE AGENTS FUNCTIONALIZED BY STIMULI-RESPONSIVE CONJUGATION WITH DNA-NANOSTRUCTURES, METHODS OF THEIR PREPARATIONS, COMPOSITIONS COMPRISING THEM AND USES THEREOF

(71) Applicant: Yissum Research Development Company of The Hebrew University of Jerusalem Ltd., Jerusalem 9139002 (IL)

(72) Inventors: Itamar WILLNER, Mevaseret Zion (IL); Eli PIKARSKY, Jerusalem (IL); Rachel NECHUSHTAI, Motza I’llit (IL); Yang-Sung SOHN, Modiin (IL); Pu ZHANG, Jerusalem (IL); Yu OUYANG, Jerusalem (IL)

(73) Assignee: Yissum Research Development Company of The Hebrew University of Jerusalem Ltd., Jerusalem (IL)

(21) Appl. No.: 18/272,579

(22) PCT Filed: Jan. 17, 2022
(86) PCT No.: PCT/IL2022/050069

§ 371 Date: Jul. 16, 2023

Related U.S. Application Data
(60) Provisional application No. 63/138,489, filed on Jan. 17, 2021.

(51) Int. Cl. A61K 9/51

(52) U.S. Cl. CPC A61K 9/5146; A61K 9/5192

(57) ABSTRACT

The present invention provides nanoparticle carriers of active agents which are functionalized by stimuli-responsive conjugation with DNA-nano structures, methods and processes for their preparation, compositions comprising them and uses thereof in methods of treating diseases, disorders and symptoms thereof.

### BACKGROUND OF THE INVENTION

Metal organic framework particles (MOFs) find growing interest as drug carrying materials due to their high porosity and high drug loading capacity, their low toxicity, their cell permeability, and the ease to modify the particles for biomedical applications. Indeed, surface-modified MOFs were used as gated carriers for the triggered release of drugs. Different triggers, such as pH, redox-agents, enzymes or light-responsive units were applied as stimuli-responsive gates of drug-loaded MOF particles for controlled release of the drugs. One specific sub-class of stimuli-responsive nano-metal organic framework nanoparticles (NMOFs) included nucleic acid-functionalized stimuli-responsive drug-loaded nucleic acid-modified NMOFs.

In these systems, the switchable triggered reconfiguration or degradation of DNA nanostructures associated with the NMOFs were used to gate (lock/unlock) the dye/drug-loaded NMOFs. Different stimuli-responsive DNA nanostructures were used to unlock the NMOFs including the use of pH-induced formation and dissociation of i-motif or triplex structures, the formation of G-quadruplexes and their separation, the separation of nucleic acid locks through the formation of aptamer-ligand complexes or by microRNA (miRNA) triggering biomarkers, and the catalytic degradation of nucleic acid locks by DNAzymes or enzymes, such as endonucleases or exonucleases. Also, the coating of NMOFs with stimuli-responsive hydrogel layers were used to unlock the NMOFs and to stimulate the release of drug loads.

Major efforts are directed towards the development of drug-loaded nano- or micro-carriers being unlocked by biological biomarkers. Such systems may act as autonomous sense-and-treat carriers for biomedical applications. For example, glucose-triggered insulin-loaded NMOFs or microcapsules were reported as autonomous glucose-guided insulin release carriers. miRNAs are short sequence-specific nucleic acids (19-30 base containing sequences) and their up-regulation and down-regulation affect cellular processes, such as proliferation and apoptosis. Links between specific miRNAs and different diseases, such as cancer, are established. Accordingly, substantial efforts to develop miRNA sensing platforms were developed. Particularly, the use of miRNAs as triggering biomarkers to release drugs from carriers provides a means to develop sense-and-treat systems. The miRNAs appear in nature in relatively low quantities, introducing difficulties to design sensors, to image, and to use the oligonucleotides as unlocking trigger of drug carriers. To overcome these difficulties, different amplification machineries to regenerate the miRNAs by enzymes or DNAzymes were reported, and sensitive analytical tools including plasmonic particles or sensitive spectroscopic means, such as surface enhanced Raman spectroscopy were introduced.

The information encoded in nucleic acids provides versatile means to design complex two-dimensional and three-dimensional nanostructures. Two-dimensional networks including three- or four-arm crossover junctions, assembly of interlocked DNA rings (catenanes), the self-assembly of two-dimensional or three-dimensional origami nanostructures, and the self-organization of three-dimensional tetrahedra DNA nanostructures were developed. The DNA tetrahedra reveals several important features for sensing and cellular biomedical applications. The DNA tetrahedra reveal high-stability, excellent cell permeability and low cytotoxicity. In addition, functional nucleic acids such as aptamers or DNAzymes can be integrated in the DNA tetrahedra structures, thus allowing the targeting of the tetrahedra to cells and their modification with catalytic units or fluorescent probes. Indeed, DNA tetrahedra were functionalized with plasmonic nanoparticles, and the effect of auxiliary triggers on the plasmonic properties of the hybrid structures were used to develop sensors. In addition, the cages of DNA tetrahedra were modified with fluorophore-quencher units, and the miRNA-guided distortion of the nanostructures was used for the multiplexed analysis of miRNAs. The conjugation of DNA tetrahedra with drug nanocarriers, such as NMOFs, is anticipated to combine the unique properties of the two elements comprising the hybrids, and to introduce hybrid nanostructures of superior functionalities for biomedical applications.

The present invention introduces hybrid nanostructures comprising stimuli-responsive DNA tetrahedra/metal organic conjugated frameworks that provide a “sense-and-treat” functional drug carriers.

### SUMMARY OF THE INVENTION

The invention thus provides a nanoparticle loaded with at least one active agent; wherein said at least one active agent is being locked within said particle by at least one DNA-nanostructure attached to said particle via at least one stimuli-responsive nucleic acid-based bridging unit.

In some embodiments, said DNA-nanostructure is selected from a four-way junction DNA, DNA-tile, DNA-crystal, DNA-cage, DNA-brick, DNA-polygonal structure, DNA-wireframe, DNA-tweezers, DNA-amphiphile, tetrahedra DNA, and any combination thereof.

When referring to “at least one stimuli-responsive nucleic acid-based bridging unit” (i.e. the bridging unit is based on at least one stimuli-responsive nucleic acid sequence), it should be understood to encompass a linker/conjugate/ligand that connects said nanoparticle loaded with said at least one active agent and said at least one DNA-nanostructure, thereby locking said loaded at least one active agent within said nanoparticle. Said linker/conjugate/ligand comprises at least one stimuli-responsive nucleic acid sequence. Once said at least one stimuli-responsive nucleic acid is subjected to said at least one stimulus, said at least one nucleic acid sequence reacts thereto, thus allows the unlocking of said at least one active agent loaded within said nanoparticle.

In other embodiments, said at least one stimuli-responsive nucleic acid based bridging unit comprises at least one stimuli-responsive sequence selected from a hairpin strand, a pH-responsive strands, a DNAzyme, a G-quadruplex, a stem-loop strand, an aptamer, a pathogen-specific sequence, microRNA, catalytically degradable sequence and any combinations thereof.

In other embodiments, said at least one stimuli-responsive nucleic acid based bridging unit further comprises a complementary sequence.

In other embodiments, said at least one DNA-nanostructure further comprises at least one targeting nucleic acid-based unit. In other embodiments, said at least one nanoparticle further comprises at least one targeting nucleic acid-based unit.

When referring to “least one targeting nucleic acid-based unit” (i.e. the targeting unit is based on at least one targeting nucleic acid sequence), it should be understood to encompass a nucleic acid ligand that comprises at least one targeting nucleic acid sequence. Said at least one targeting nucleic acid sequence is selected so as to have an association with a specific target, such as for example a cell or tissue (diseased or healthy for the purpose of preventing a disease), a phenotype, a pathogen, a virus, a microbial entity, a combination thereof, and so forth.

Said nanoparticle of the present invention is capable of being loaded within its structure (for example within pores or voids of said structure) with at least one active agent. In some embodiments, said nanoparticle is a metal organic nanoparticle (also known as metal-organic framework—MOF).

Metal-organic frameworks (MOFs) are a class of compounds consisting of metal ions or clusters coordinated to organic ligands to form one-, two-, or three-dimensional porous structures. MOF is a coordination network with organic ligands containing potential voids.

In other embodiments, said at least one targeting nucleic acid-based unit comprises a targeting sequence selected from a hairpin strand, a pH-responsive strands, a DNAzyme, a G-quadruplex, a stem-loop strand, an aptamer, a pathogen-specific sequence, microRNA, catalytically degradable sequence and any combinations thereof.

In other embodiments, said stimuli is selected from pH, radiation, light, temperature, gas, salt, metal, chemical reducing agent, carbohydrate, enzyme, magnetic field, ultrasonic or microwave agitation and any combinations thereof.

In other embodiments, said at least one active agent is selected from a drug, a pro-drug, a labeling agent, a hormone, a reactive compound capable of forming at least one stimulus, a nucleic acid sequence, a steroid, a sensor, a transistor, a radioactive agent and any combinations thereof.

The invention provides a composition comprising at least one nanoparticle as disclosed herein above and below. In some embodiments, said composition is a pharmaceutical composition.

The invention also provides a process for the preparation of a nanoparticle loaded with at least one active agent; wherein said at least one active agent is being locked within said particle by at least one DNA-nanostructure attached to said particle via at least one stimuli-responsive nucleic acid-based bridging unit; said process comprising the steps of: (a) attaching at least one first nucleic acid sequence to a nanoparticle; thereby providing a modified nanoparticle; (b) loading said modified meal organic nanoparticle with at least one active agent; thereby providing a loaded modified nanoparticle; (c) hybridizing said loaded modified nanoparticle with at least one DNA-nanostructure comprising at least one second nucleic acid sequence; wherein at least one of said first and second nucleic acid sequence is a stimuli-responsive sequence; thereby forming said at least one stimuli-responsive nucleic acid-based bridging unit attaching said at least one DNA-nanostructure with said nanoparticle and locking said at least one active agent within said particle.

In some embodiments, said first nucleic acid sequence is a stimuli-responsive sequence. In other embodiments, said second nucleic acid sequence is a stimuli-responsive sequence.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1D. (1A) Synthesis of nucleic acid (1)-functionalized UiO-66 NMOFs. (1B) SEM image of the (1)-modified UiO-66 NMOFs. The inset is the STEM image of (1)-modified UiO-66 NMOFs (Scale bar in the inset is 200 nm). (1C) Schematic assembly of the DNA tetrahedra using the strands S₁-S₄ (for detailed sequences see experimental section). The tether (a) is complementary to strand (1)-modified NMOFs and used to link the tetrahedra units to the NMOFs, through hybridization. Tether (b) consists of the AS1411 aptamer that guides the hybrids tetrahedra/NMOFs to nucleolin receptors associated with MDA-MB-231 breast cancer cells. (1D) Electrophoretic separation following the stepwise assembly of the tetrahedra structure. Panel (I)-Electrophoresis on 1% agarose gel: Lane 1-Ladder; Lane 2-S₁; Lane 3-S₁+S₂; Lane 4-S₁+S₂+S₃; Lane 5-S₁+S₂+S₃+S₄. Panel (II)-Electrophoresis on 12% PAGE gel: Lane 1-Ladder; Lane 2-S₁; Lane 3-S₁+S₂; Lane 4-S₁+S₂+S₃; Lane 5-S₁+S₂+S₃+S₄.

FIGS. 2A-2C. (2A) Assembly of the DOX-loaded T/NMOFs hybrids and the pH-induced release of the loads, through the formation of the i-motif structures, pH=5.5. (2B) Time-dependent release of DOX from the DOX-loaded T/NMOFs: (i) At pH=5.5; (ii) pH=7.2. (2C) Fluorescence spectra of the released DOX from the T/NMOFs upon pH=5.5 at different time-intervals: (a) 0 min; (b) 10 mins; (c) 20 mins; (d) 30 mins; (e) 40 mins; (f) 100 mins. Error bars derived from N=3 experiments.

FIGS. 3A-3D. (3A) Confocal microscopy images comparing the permeation of the DOX-loaded T/NMOFs, and of the control system composed of DOX-loaded (1)/(2) duplex-locked NMOFs into MDA-MB-231 and MCF-10A cells. Panel I-The DOX-loaded T/NMOFs into MDA-MB-231 cells. Panel II-The DOX-loaded T/NMOFs into MCF-10A cells. Panel III-The duplex (1)/(2)-locked DOX-loaded NMOFs into MDA-MB-231 cells. Panel IV-The (1)/(2) duplex-locked DOX-loaded NMOFs into MCF-10A cells. (3B) Quantitative analysis of the confocal microscopy images relating the fluorescence intensity of DOX to the normalized content of cells in the form of a “bar” presentation. (3C) Schematic targeted penetration of the DOX-loaded T/NMOFs into the MDA-MB-231 cancer cells and pH-induced unlocking of the NMOFs and release of DOX in the cytoplasm. (3D) Cytotoxicity of the DOX-loaded T/NMOFs and DOX-loaded (1)/(2) duplex-gated NMOFs towards MDA-MB-231 cells and non-malignant MCF-10A epithelial breast cells. Panel I-Nontreated cells. Panel II-Treatment of the cells with non-loaded pH-responsive T/NMOFs. Panel III-Treatment of the cells with pH-responsive DOX-loaded (1)/(2) duplex-locked NMOFs. Panel IV-Treatment of the cells with pH-responsive DOX-loaded T/NMOFs. Cells treated with the respective NMOFs for five days. 30 μg/mL NMOFs included an identical content of DOX (0.035 mg/per mg NMOFs). Error bars derived from N=3 experiments.

FIGS. 4A-4D. (4A) Path (I)-Preparation of miRNAs-responsive T_{A}/NMOFs or T_{B}/NMOFs hybrids loaded with a dye or anti-cancer drug, and the schematic miRNA-stimulated release of the loads. Path (II)-Design of the miRNAs-responsive T_{A}/NMOFs or T_{B}/NMOFs hybrids loaded with dyes/anti-cancer drugs that allows the Exo III regeneration of the miRNAs for the guided unlocking of the tetrahedra locks and the release of the loads. (4B) Time-dependent fluorescence changes upon the release of Rhodamine 6G from the miRNA-21 responsive T_{A}/NMOFs loaded with the dye using different concentrations of miRNA-21: (i) 1.0 μM; (ii) 500 nM; (iii) 0 nM. (4C) Fluorescence studies demonstrating the selective miRNA-guided release of the dyes from miRNA-21 responsive T_{A}/NMOFs loaded with Rhodamine 6G and from the miRNA-155 responsive T_{B}/NMOFs loaded with fluorescein: Panel-(i) Fluorescence spectra of Rhodamine 6G released from the miRNA-21 responsive T_{A}/NMOFs in the presence of: (a) miRNA-21; (b) miRNA-155; (c) miRNA-145; (d) no miRNA. Panel-(ii) Fluorescence spectra of fluorescein released from the miRNA-155 responsive T_{B}/NMOFs in the presence of: (a) miRNA-155; (b) miRNA-21; (c) miRNA-145; (d) no miRNA. Spectra recorded after a release time-interval of 40 minutes, concentrations of the different miRNAs corresponded to 1 μM. (4D) Time-dependent fluorescence changes upon the miRNA-21 triggered release of DOX from the miRNA-21 responsive T_{A}/NMOFs using different concentrations of miRNA-21 corresponding to (i) 1.0 μM; (ii) 500 nM; (iii) 0 nM. Error bars derived from N=3 experiments.

FIGS. 5A-5B. (5A) Multiplexed miRNA-21 and miRNA-155 guided release of fluorescein (λₑₘ=520 nm) and Rhodamine 6G (λₑₘ=550 nm) from a mixture of Rhodamine 6G-loaded miRNA-21 responsive T_{A}/NMOFs and fluorescein-loaded miRNA-155 responsive T_{B}/NMOFs in the presence of (a) miRNA-21; (b) miRNA-155; (c) miRNA-21 and miRNA-155. (5B) Multiplexed miRNA-21 and miRNA-155 guided release of DOX (λₑₘ=590 nm) and CPT (λₑₘ=430 nm) from a mixture of DOX-loaded miRNA-21 responsive T_{A}/NMOFs and CPT-loaded miRNA-155 responsive T_{B}/NMOFs in the presence of (a) miRNA-21; (b) miRNA-155; (c) miRNA-21 and miRNA-155. The concentration of the addition of miRNA-21 and miRNA-155 corresponding to 1.0 μM, respectively. Release examined in a mixture consisting of 0.5 mgmL⁻¹ of each of the T_{A}/NMOFs and T_{B}/NMOFs.

FIGS. 6A-6C. (6A) Time-dependent release of DOX from the miRNA-21 responsive T_{A}/NMOFs, 0.5 mg·mL⁻¹, in the presence of miRNA-21, 200 nM: (i) In the presence of added Exo III, 1 U·μL⁻¹. (ii) In the absence of added Exo III. (6B) Fluorescence values of released DOX recorded after a fixed time interval of 90 minutes upon subjecting the miRNA-21 responsive T_{A}/NMOFs loaded with DOX, 0.5 mg·mL⁻¹, to variable concentrations of miRNA-21: (i) In the presence of Exo III, 1 U·μL⁻¹. (ii) In the absence of added Exo III. (6C) Time-dependent release of CPT from the miRNA-155 responsive T_{B}/NMOFs, 0.5 mg·L⁻¹, in the presence of miRNA-155, 200 nM: (i) In the presence of added Exo III, 1 U·μL⁻¹. (ii) In the absence of added Exo III. Error bars derived from N=3 experiments.

FIGS. 7A-7C. Multiplexed miRNA-triggered release of DOX and/or CPT from a mixture of miRNA-21 responsive DOX-loaded T_{A}/NMOFs and miRNA-155 responsive CPT-loaded T_{B}/NMOFs: Panel A-Fluorescence spectra of CPT (a) and DOX (b) upon treating the mixture with miRNA-21, 1 μM. Panel B-Fluorescence spectra of CPT (a) and DOX (b) upon treating the mixture with miRNA-155, 1 μM. Panel C-Fluorescence spectra of CPT (a) and DOX (b) upon treating the mixture with miRNA-21, 1 μM and miRNA-155, 1 μM. Release examined in a mixture consisting of 0.5 mg·mL⁻¹ of each of the T_{A}/NMOFs and T_{B}/NMOFs, in the presence of Exo III, 1 U·μL⁻¹.

FIGS. 8A-8D. (8A) Schematic structures of the miRNA-21 responsive DOX-loaded NMOFs gated by the (2)/(5)-duplex units and by the tetrahedra A, T_{A}. (8B) Confocal microscopy images corresponding to: Panel I-MDA-MB-231 breast cancer cells treated with the DOX-loaded (2)/(5) duplex-gated NMOFs. Panel II-MDA-MB-231 breast cancer cells treated with the DOX-loaded T_{A}/NMOFs. Panel III-MCF-10A epithelial cells treated with the DOX-loaded (2)/(5)-duplex gated NMOFs. Panel IV-MCF-10A epithelial cells treated with the DOX-loaded T_{A}/NMOFs. Cells were subjected to 60 μg·mL⁻¹ of NMOFs for a time interval of 6 hours. (8C) Normalized fluorescence intensities of DOX associated with the respective DOX-containing cell cultures (1.75-fold enhanced permeation of the T_{A}/NMOFs into the MDA-MB-231 cancer cell as compared to the (2)/(5)-duplex gated NMOFs into the MDA-MB-231 cells is observed). (8D) Cytotoxicity NMOFs towards MCF-10A epithelial breast cells, MDA-MB-231 breast cancer cells and HepG2 liver cancer cells and appropriate control systems. Panel I-Non-treated cells. Panel II-Treatment of the cells with non-loaded miRNA-21 responsive T_{A}/NMOFs. Panel III-Treatment of the cells with miRNA-21 responsive DOX-loaded (2)/(5)-duplex gated NMOFs. Panel IV-Treatment of the cells with miRNA-21 responsive DOX-loaded T_{A}/NMOFs. Cells viabilities were recorded after a time interval of three days subjecting the respective cells with 60 μg·mL⁻¹ of NMOFs. Error bars derived from N=3 experiments.

FIGS. 9A-9C. (9A) Synthesis of the ligand 2-azido-terephthalic acid. (9B) 1H NMR spectrum of ligand 2-azido-terephthalic acid in Chloroform-d. (9C) FTIR spectrum of the ligand 2-azido-terephthalic acid.

FIG. 10. Evaluation of the loading of DNA anchor (1) associated with the NMOFs. 4.5 mg of NMOFs were introduced into a solution of 2 mL that contained 180 nmols of DNA anchor (1). The absorption spectrum of the solution was recorded prior to the addition of the NMOFs. After reaction of the NMOFs with DNA anchor (1), the NMOFs were precipitated, and the absorption spectrum of the supernatant was recorded to evaluated the concentration of unreacted DNA anchor (1). The NMOFs were washed twice with water and the spectra of the washing solution were recorded, spectra (i) and (ii), respectively. The concentrations of (1) in the rinsing solution were added to the primary concentrations of non-reacted (1) and the total concentration of residual (1) subtracted from the initial concentration of (1), reacted with the NMOFs to quantitatively evaluate the loading of (1) on the NMOFs. Using this procedure, the loading of (1) corresponded to 8.56 nmols per 1 mg of NMOFs.

FIGS. 11A-11C. (11A) XRD pattern of the UiO-66 NMOFs. (11B) Dynamic light scattering spectrum of the NMOFs. (11C) The zeta-potential of NMOFs. Panel I-UiO-66 MOFs; Panel II-DNA anchor (1) modified-UiO-66 MOFs; Panel III-DNA anchor (1) modified-DOX loaded-UiO-66 MOFs; Panel IV-DNA (1)/DNA tetrahedra-modified DOX-loaded UiO-66 NMOFs.

FIG. 12. Fluorescence spectra of released DOX from T/NMOFs, 0.1 mg, at different time intervals of release: (a) 0 min; (b) 10 mins; (c) 20mins; (d) 30 mins; (e) 40 mins; (f) 100 mins. Release was performed at pH=7.2 and corresponded to the background release of DOX from the NMOFs.

FIGS. 13A-13C. (13A) Fluorescence spectra of DOX at different concentrations (i) 0.1 μM; (ii) 1 μM; (iii) 5 μM; (iv) 20 μM; (v) 25 μM. (13B) The calibration curve corresponding to the fluorescence intensities as a function of the concentration of DOX. (C) Evaluation of the loading of DOX on the T/NMOFs. 0.1 mg of T/NMOFs were introduced in 1 mL of a 14.5 mM DOX solution. The mixture was stirred for 12 hours. Afterwards, the NMOFs were precipitated, and the fluorescence spectrum of the supernatant solution was recorded, FIG. 13C), and using the calibration curve shown in FIG. 13B), the loading of DOX corresponded to 65 nmols per 1 mg of NMOFs

FIGS. 14A-14D. (14A) Panel I-The calibration curve corresponding to the fluorescence intensities as a function of the concentration of Rhodamine 6G. Panel II-Evaluation of the loading of Rhodamine 6G on the T_{A}/NMOFs. 1 mg of T_{A}/NMOFs were introduced in 1 mL of a 0.216 mM Rhodamine 6G solution. The mixture was stirred for 12 hours. Afterwards, the T_{A}/NMOFs were precipitated and the fluorescence spectrum of the supernatant solution was recorded, FIG. 14A), panel II, and using the calibration curve shown in FIG. 14A), panel I, the loading of Rhodamine 6G corresponded to 68 nmols per 1 mg of NMOFs. (14B) Panel I-The calibration curve corresponding to the fluorescence intensities as a function of the concentration of fluorescein. Panel II-Evaluation of the loading of fluorescein on the T_{B}/NMOFs. 1 mg of T_{B}/NMOFs were introduced in 1 mL of a 0.12 mM fluorescein solution. The mixture was stirred for 12 hours. Afterwards, the T_{B}/NMOFs were precipitated and the fluorescence spectrum of the supernatant solution was recorded, FIG. 14B), panel II, and using the calibration curve shown in FIG. 14B), panel I, the loading of fluorescein corresponded to 60 nmols per 1 mg of T_{B}/NMOFs. (14C) Panel I-Evaluation of the loading of DOX on the T_{A}/NMOFs. 1 mg of T_{A}/NMOFs were introduced in 1.5 mL of a 14.5 mM DOX solution. The mixture was stirred for 12 hours. Afterwards, the T_{A}/NMOFs were precipitated and the fluorescence spectrum of the supernatant solution was recorded, FIG. 14C), panel I, and using the calibration curve shown in FIG. 13B), the loading of DOX corresponded to 66 nmols per 1 mg of T_{A}/NMOFs. (14D) Panel I-The calibration curve corresponding to the CPT intensities as a function of the concentration of CPT. Panel II-Evaluation of the loading of CPT on the T_{B}/NMOFs. 1 mg of T_{B}/NMOFs were introduced in 1 mL of a 0.193 mM CPT solution. The mixture was stirred for 12 hours. Afterwards, the T_{B}/NMOFs were precipitated and the fluorescence spectrum of the supernatant solution was recorded, FIG. 14D), panel II, and using the calibration curve shown in FIG. 14D), panel I, the loading of CPT corresponded to 73 nmols per 1 mg of T_{B}/NMOFs.

FIGS. 15A-15C. (15A) Time-dependent fluorescence changes upon the miRNA-21 triggered release of fluorescein from fluorescein-loaded T_{B}/NMOFs using different concentrations of miRNA-21: (i) 1.0 μM; (ii) 500 nM; (iii) 0 nM. (15B) Fluorescence spectra of Rhodamine 6G released from the Rhodamine 6G-loaded miRNA-21 responsive T_{A}/NMOFs at fixed time interval of 100 minutes with the concentration of miRNA-21 corresponding to: (i) 1.0 μM; (ii) 500 nM; (iii) 0 nM. (15C) Fluorescence spectra of fluorescein released from the miRNA-155 responsive fluorescein-loaded T_{B}/NMOFs at fixed time interval of 100 minutes with the concentration of miRNA-155 corresponding to: (i) 1.0 μM; (ii) 500 nM; (iii) 0 nM.

FIGS. 16A-16C. (16A) Time-dependent fluorescence changes upon the miRNA-155 triggered release of CPT from CPT-loaded T_{B}/NMOFs using different concentrations of miRNA-155: (i) 1.0 μM; (ii) 500 nM; (iii) 0 nM. (16B) Fluorescence spectra of DOX released from the DOX-loaded miRNA-21 responsive T_{A}/NMOFs at fixed time interval of 100 minutes with the concentration of miRNA-21 corresponding to: (i) 1.0 μM; (ii) 500 nM; (iii) 0 nM. (16C) Fluorescence spectra of CPT released from the CPT-loaded miRNA-155 responsive T_{B}/NMOFs at fixed time interval of 100 minutes with the concentration of miRNA-155 corresponding to: (i) 1.0 μM; (ii) 500 nM; (iii) 0 nM.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

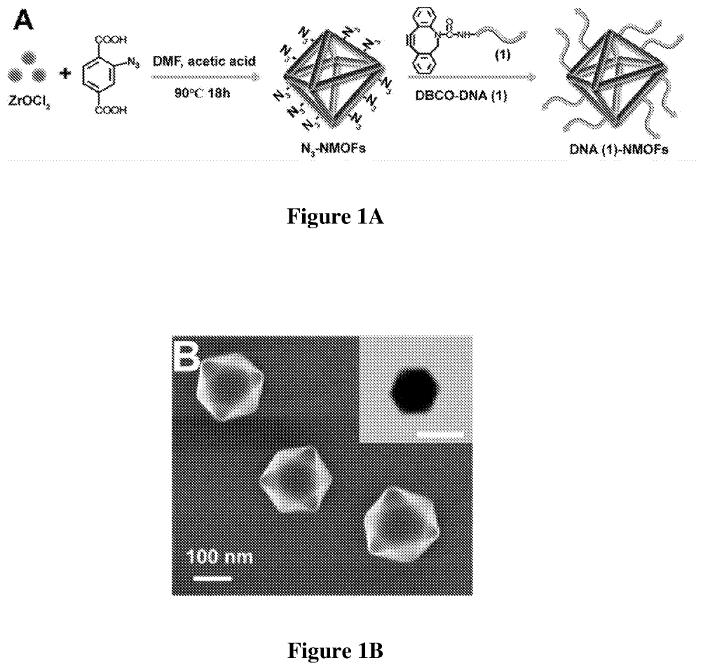

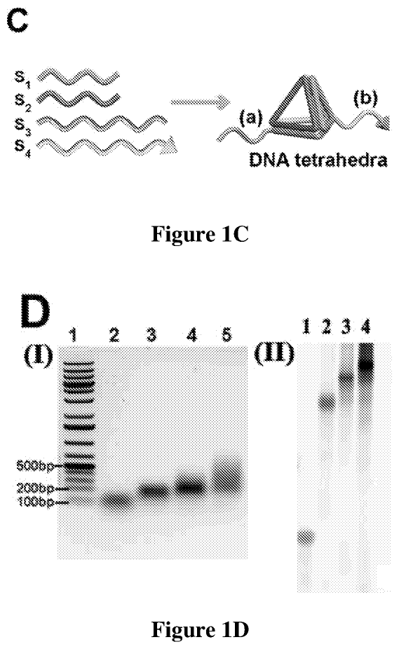

The ligand 2-azido-terephthalic acid was prepared from 2-aminoterephthalic acid as reported previously (Trigo-López, M.; Barrio-Manso, J. L.; Serna, F.; García, F. C.; García, J. M. Crosslinked Aromatic Polyamides: A Further Step in High-Performance Materials. Macromol. Chem. Phys. 2013, 214, 2223-2231). Azide-modified UiO-66 nanoscaled metal-organic framework nanoparticles, NMOFs, were prepared by the reaction between 2-azido-terephthalic acid and ZnOCl₂. The resulting NMOFs were covalently linked to DBCO-functionalized nucleic acid (1), (DBCO=dibenzocyclooctyne), using the click chemistry principle to yield the (1)-functionalized NMOFs, FIG. 1(A). The coverage of the NMOFs with (1) was evaluated spectroscopically, to be 8.56 nmole per 1 mg of the NMOFs. FIG. 1(B) shows the SEM and STEM images of the resulting (1)-modified NMOFs. Bipyramidal nanoparticles, exhibiting a 200 nm cross-section, are formed. In addition, powder X-ray diffraction (XRD) and dynamic light-scattering (DLS) experiments were performed. The zeta-potential of the NMOFs corresponded to ca. −10 mV, prior to the modification with (1), and −45 mV after modification with (1). The DNA tetrahedra were prepared by the stepwise self-assembly of the four nucleic acids (S₁), (S₂), (S₃) and (S₄), FIG. 1(C). The purity of the DNA tetrahedra was confirmed by electrophoretic separation of the intermediate structures, FIG. 1(D), panels I and II. The DNA tetrahedra were engineered to include two free tethers, the tether (a) includes the complementary stimuli-responsive sequence that hybridizes with the nucleic acid, anchor strand (1), associated with the NMOFs. The second tether (b) includes the AS1411 aptamer sequence that binds to the nucleolin receptor associated with MDA-MB-231 breast cancer cells.

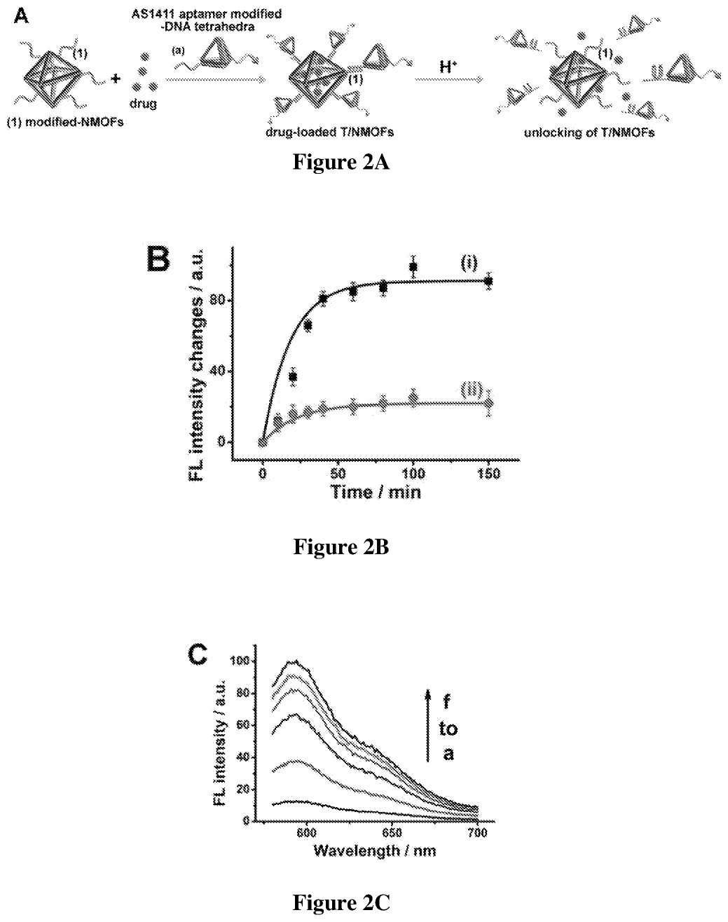

The preparation of the stimuli (pH)-responsive drug (or dye)-loaded DNA tetrahedra-locked NMOFs (T/NMOFs) and the mechanism to unlock and release the loads are displayed in FIG. 2(A). The (1)-modified NMOFs were loaded with the doxorubicin (DOX) drug, and the NMOFs were locked with the tetrahedra through the hybridization of the tethers (a) with (1). The loading of the tetrahedra on the NMOFs was evaluated spectroscopically to be 6.5 nmols per mg NMOFs. The zeta-potential of the T/NMOFs and DOX-loaded T/NMOFs corresponded to −20 mV and −30 mV, respectively. The tether (a) associated with the NMOFs and hybridized with (1) was engineered to include the cytosine-rich sequence that yields at acid pH-values, the i-motif structure. As a result, at pH=5.5, the tetrahedra are dissociated from the NMOFs resulting in the unlocking of the particles and the release of the loads. FIG. 2(B), curve (i) depicts the time-dependent release of DOX upon subjecting the T/NMOFs to pH=5.5. FIG. 2(B), curve (ii) shows the time-dependent release of DOX from the T/NMOFs at pH=7.2. Only a residual release of DOX is observed, that is attributed to the release of DOX from imperfectly locked sites associated with the NMOFs. The results confirm that the release of DOX is stimulated by the acidic pH that dissociates the tetrahedra from the loaded NMOFs. FIG. 2(C) shows the fluorescence spectra of the released DOX at different time intervals after unlocking the particles at to pH=5.5 and pH=7.2, respectively. After ca. 60 minutes, the fluorescence of the released DOX reached saturation, implying that all the loaded DOX was released. From the saturated fluorescence of the released DOX, and using an appropriate calibration curve, it was evaluated that the loading of DOX to be 65 nmoles of DOX per mg of NMOFs.

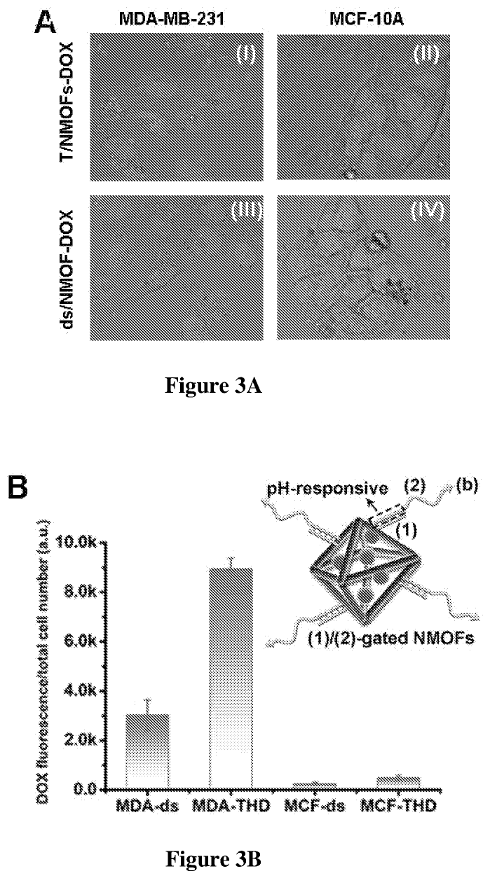

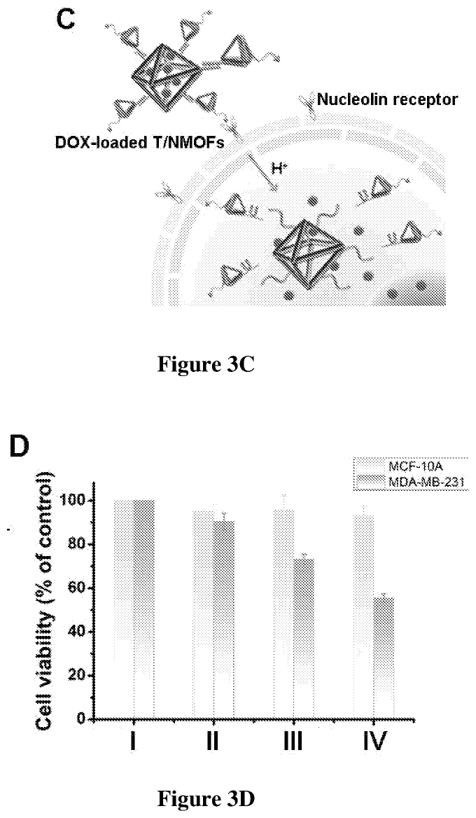

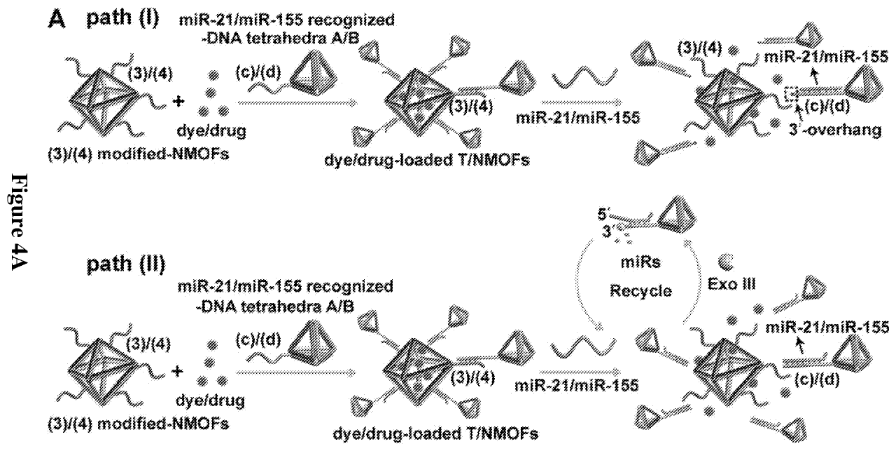

In the next step, the cell permeation capacity and the cytotoxicity of the DOX-loaded T/NMOFs towards MDA-MB-231 breast cancer cells, in comparison to non-malignant MCF-10A breast epithelial cells, were examined, FIG. 3. In addition, the cell permeation capacity of the T/NMOFs and their cytotoxicity were compared to a control system comprising of DOX-loaded NMOFs locked by duplex gates (1)/(2) where (1) is the anchoring strand associated with the NMOFs and the strand (2) is composed of the pH-responsive sequence, complementary to (1), extended by the AS1411 aptamer sequence (b) (The structure of the duplex (1)/(2)-gated NMOFs shows in FIG. 3(B), inset). FIG. 3(A), panels I and II show the confocal fluorescence images of the DOX-loaded T/NMOFs associated with MDA-MB-231 cancer cells and the non-malignant MCF-10A breast epithelial cells, respectively. FIG. 3, panel III and IV show the reference systems composed of the confocal fluorescence images of the DOX-loaded (1)/(2)-duplex locked NMOFs associated with the MDA-MB-231 cells and the MCF-10A cells, respectively. The integrated fluorescence intensities of the respective systems, in the form of a “bar” presentation is shown in FIG. 3(B). The results reveal effective permeation of the NMOFs into the MDA-MB-231 cancer cells and very inefficient permeation into the non-malignant MCF-10A breast epithelial cells. Furthermore, the permeation of the DOX- loaded T/NMOFs into the MDA-MB-231 cells is three-fold more efficient as compared to the reference system composed of the (1)/(2)-duplex NMOFs loaded with DOX. The improved permeation of the NMOFs into the MDA-MB-231 cells, as compared to the MCF-10A cells is attributed to the AS1411 aptamer guided binding of the NMOFs to the nucleolin receptor sites present in the cancer cells, yet absent in the non-malignant MCF-10A breast epithelial cells. The improved permeation of the T/NMOFs into the MDA-MB-231 cells, as compared to the (1)/(2)-duplex NMOFs, is attributed to the DNA-tetrahedra facilitated permeation into cells. The permeation features of the pH-responsive tetrahedra-functionalized NMOFs with the cancer cells, the pH-induced release of DOX from the NMOFs, and the resulting cytotoxicity of the released drug towards the cancer cells are schematically presented in FIG. 3(C). The cytotoxicity of the DOX-loaded pH-responsive T/NMOFs towards MDA-MB-231 breast cancer cells, and non-malignant MCF-10A breast epithelial cells is summarized in FIG. 3(D) and compared to the reference system composed of the DOX-loaded (1)/(2)-duplex locked NMOFs. The respective cell cultures were treated for five days with identical amounts of DOX-loaded NMOFs consisting of T/NMOFs and the reference (1)/(2)-gated NMOFs carriers. For comparison, the cell viability of non-treated cells is shown in FIG. 3(D), panel I. The results in panel II show that the non-loaded pH-responsive T/NMOFs have no effect on the viability of the MDA-MB-231 cells and MCF-10A cells. After a time-interval of five days, ca. 50% cell death of the MDA-MB-231 cells is observed whereas the non-malignant MCF-10A breast epithelial cells were almost unaffected <10% cell death upon treating the cells with the DOX-loaded T/NMOFs, FIG. 3(D), panel IV. In turn, in the reference system composed of the (1)/(2)-gated drug-loaded NMOFs, only 30% cell motility is detected after this time interval, FIG. 3(B), panel III. These results and the selective cytotoxicity towards the cancer cells are consisted with the pH-responsive triggered drug release mechanism and the improved permeation of the T/NMOFs into the cancer cells. The lack of nucleolin receptor sites in epithelial cells (to be targeted by the AS1411 aptamer), the inefficient permeation of the NMOFs into the non-malignant MCF-10A breast epithelial cells, and the lack of acidic conditions in the epithelial cells lead to unlock the carriers to the lack of cytotoxicity towards the MCF-10A breast epithelial cells. The improved cytotoxicity of the T/NMOFs towards the cancer cells is attributed to the enhanced permeation of the T/NMOFs hybrids into the cancer cells, as compared to the (1)/(2)-gated drug carriers.

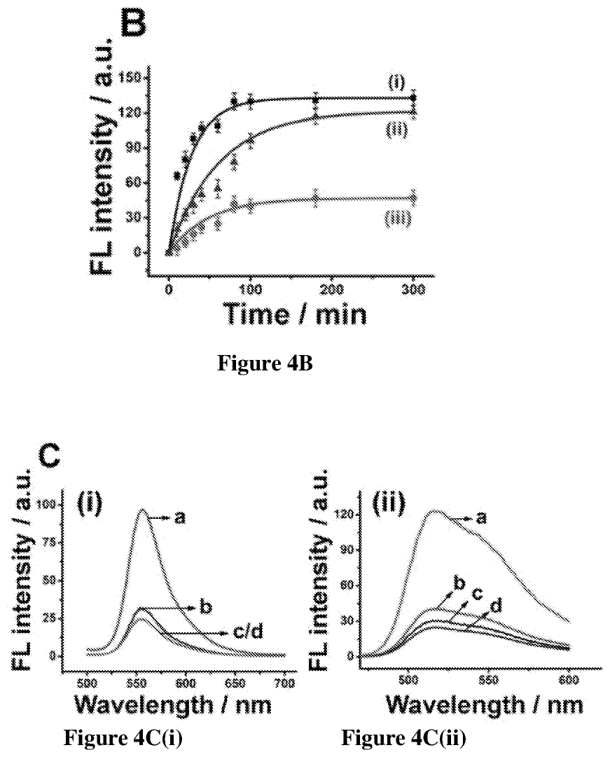

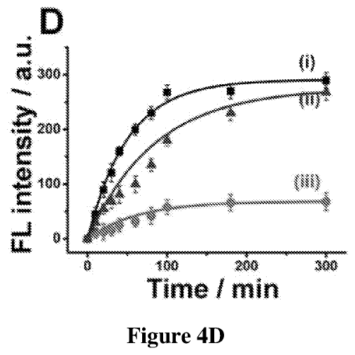

The design of stimuli-responsive T/NMOFs has been extended to include miRNA-responsive drug carriers. The availability of different miRNA biomarkers for different cancer cells provides means to design tetrahedra-gated NMOFs for different miRNAs (miRNA-guided carriers for the release of drugs), and methods to design mixtures of different miRNA-responsive NMOFs carrying different drugs/dyes (for the miRNA-guided programmed release of drugs/dyes). In addition, realizing the low intracellular concentrations of miRNAs, a versatile concept to amplify the miRNAs-guided unlocking of the T/NMOFs for the programmed release of the drugs was introduced. FIG. 4(A), path (I) depicts the principle to design drug-loaded (or dye-loaded as drug models) miRNA-responsive T/NMOFs. The UiO-66 NMOFs were functionalized with the anchoring nucleic acid (3) or (4), and DNA tetrahedra A or B that include engineered tethers (c) or (d), for the gating of two different miRNA-responsive T/NMOFs (for miRNA-21 or miRNA-155) through hybridization to the (3)- or (4)-functionalized NMOFs. The (3)-modified NMOFs were loaded with DOX (or the Rhodamine 6G dye) and the (4)-modified NMOFs were loaded with the camptothecine (CPT) drug (or the fluorescein dye), and subsequently locked with the tetrahedra A or B, respectively. The loading of DOX, Rhodamine 6G, CPT and fluorescein corresponded to 66 nmols/mg NMOFs, 68 nmols/mg NMOFs, 73 nmols/mg NMOFs, and 60 nmols/mg NMOFs, respectively (For the evaluation of the loading of the respective dyes/drugs). The miRNA-21 or miRNA-155-induced displacement of the (c)/(d) tethered tetrahedra unlocked the NMOFs, resulting in the release of the respective drugs (or dyes). The duplex of (c)/miRNA-21 or (d)/miRNA-155 will not be digested by Exonuclease III, Exo III, due to the single strand tethers embedded at 3′-end of (c)/(d)). FIG. 4(B) shows the time-dependent fluorescence changes of the Rhodamine 6G-loaded tetrahedra A-locked NMOFs (T_{A}/NMOFs) upon treatment of the carrier with different concentrations of miRNA-21 corresponding to 1.0 μM (curve i), 500 nM (curve ii) and 0 nM (curve iii). Similarly, the time-dependent fluorescence changes upon unlocking the fluorescein-loaded tetrahedra B-locked NMOFs (T_{B}/NMOFs), upon subjecting the carrier to different concentrations of miRNA-155. The results shown in FIG. 4(B) reveal that as the concentrations of miRNA-21 or miRNA-155 are elevated, the rates of the released dyes are enhanced, consistent with the improved uncaging of the NMOFs as the concentrations of the miRNAs increase. The fluorescence spectra of the Rhodamine 6G and fluorescein dyes show they were released from the respective NMOFs, after a time-interval of 100 minutes, in the presence of different concentrations of miRNA-21 and miRNA-155, respectively. The release profiles of the dyes, shown in FIG. 4(B) reveals saturation levels that correspond to the complete release of the loads. The contents of the released Rhodamine 6G and fluorescein were evaluated using appropriate calibration curves and these corresponded to 68 and 60 nmoles·mg⁻¹ NMOFs, respectively, consistent with the original loading levels of the dyes. Control experiments revealed that treatment of the miRNA-21 responsive Rhodamine 6G-loaded T_{A}/NMOFs with miRNA-155 or miRNA-145 or of the miRNA-155 responsive fluorescein-loaded T_{B}/NMOFs with miRNA-21 or miRNA-145 did not lead to the release of the loads, indicating that the release of the dyes from the respective NMOFs is specific to miRNA-21, FIG. 4(C) panel (i), and miRNA-155, FIG. 4(C) panel (ii), respectively. FIG. 4(D) depicts the time-dependent fluorescence changes observed upon the release of DOX and CPT, upon subjecting the miRNA-21 responsive DOX loaded-T_{A}/NMOFs and of the miRNA-155 responsive CPT loaded-T_{B}/NMOFs upon treatment with different concentrations of miRNA-21 and miRNA-155, respectively. The release of the load is effective at a miRNA-21 concentration corresponding to 1.0 μM (curve i) and 500 nM (curve ii), the release of the drug is inefficient in the absence of miRNA-21 (curve iii). As the concentrations of miRNA-21 and miRNA-155 increase the release rates are enhanced, consistent with the improved uncaging of the NMOFs as the concentration of the respective miRNAs increase. The fluorescence spectra of the DOX and CPT show they were released from the respective NMOFs, after a time-interval of 100 minutes, in the presence of different concentrations of miRNA-21 and miRNA-155, respectively. The saturated levels of the released DOX and CPT, shown in FIG. 4(D), correspond to the complete release of the respective drugs from the carriers. The derived loading of DOX and CPT in the respective carriers were evaluated using appropriate calibration curves and these correspond to 66 and 73 nmoles·mg⁻¹ of the carriers, values that agree well with loading values upon the synthesis of NMOFs vide supra.

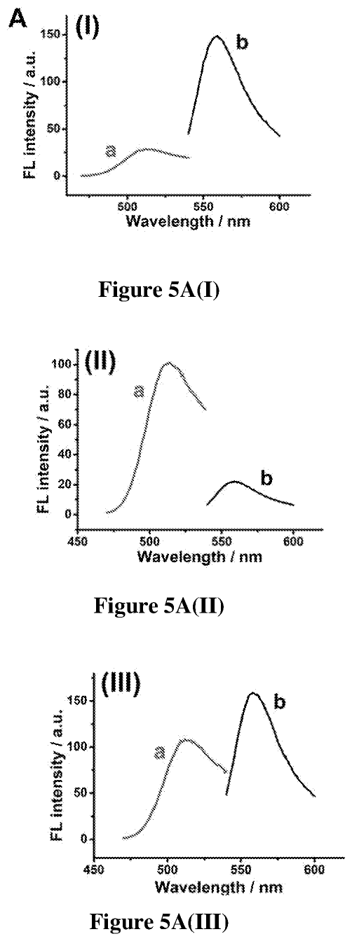

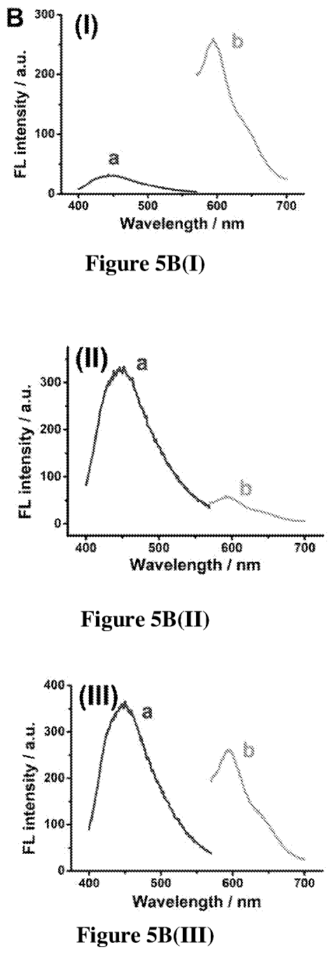

The successful release of the two different drugs (or dyes) from the two different NMOFs enables the miRNA-dictated release of the loads from the mixture of the carriers. In this experiment, the mixture of miRNA-responsive NMOFs, miRNA-21 responsive T_{A}/NMOFs and miRNA-155 responsive T_{B}/NMOFs, was loaded with different drugs (or dyes) and unlocked in the presence of two different miRNAs. FIG. 5(A) depicts the dictated unlocking of the Rhodamine 6G and fluorescein-loaded NMOFs mixture by the miRNAs. In the presence of miRNA-21, only the Rhodamine 6G-loaded T_{A}/NMOFs are unlocked, leading to the fluorescence of Rhodamine 6G (Panel I, curve b) and to the background fluorescence corresponding to the inefficient leakage of the fluorescein from the T_{B}/NMOFs (Panel I, curve a). In the presence of miRNA-155, only the fluorescein-loaded T_{B}/NMOFs are unlocked, leading to the release and fluorescence of fluorescein (Panel II, curve a) and to the background fluorescence corresponding to the inefficient leakage of the Rhodamine 6G from the T_{A}/NMOFs (Panel I, curve b). In the presence of the miRNA-21 and miRNA-155, the two NMOFs are unlocked, leading to the release of the Rhodamine 6G (curve b) and fluorescein (curve a), Panel III. Similarly, FIG. 5(B) shows the miRNA-dictated release of DOX and CPT from the mixture of DOX-loaded T_{A}/NMOFs and CPT-loaded T_{B}/NMOFs. In the presence of miRNA-21, only DOX is released, Panel I, curve b, while, in the presence of miRNA-155 only CPT is released, Panel II, curve a. Subjecting the mixture of NMOFs to miRNA-21 and miRNA-155 lead to the uncaging of the two kinds of the NMOFs and to the release of DOX (curve b) and CPT (curve a), Panel III.

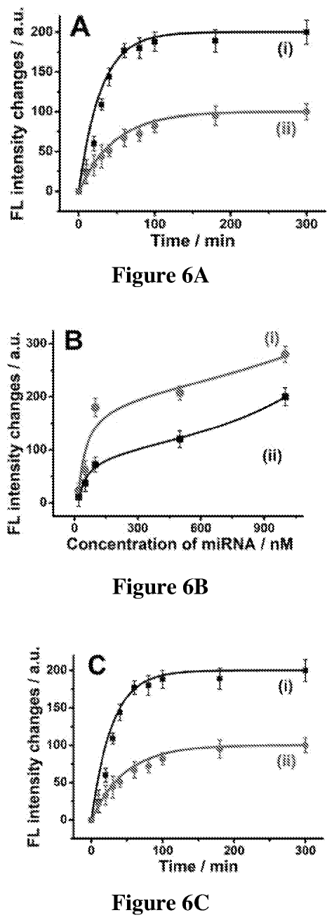

Realizing the low contents of miRNA in cells, and with the vision that the cytotoxicity of the NMOFs towards cancer cells were examined, vide infra, and means to amplify the miRNA-triggered release process were searched. FIG. 4(A), path (II), depicts the amplification cycle used to amplify the miRNA-triggered release of the loads. The NMOFs are functionalized with nucleic acid (3)/(4), and loaded with respective drug/dye, and the loads were locked by hybridization of the nucleic acid (3)/(4), with (c)/(d) tethered tetrahedra A/B, respectively. The miRNA-21 and miRNA-155 triggered unlocking of the NMOFs yields the duplex structures of miRNA-21/tether (c) upon separation of the tetrahedra A, and of the miRNA-155/tether (d) upon separation of the tetrahedra B, where the 3′-ends of the duplexes miRNA-21/(c) and the miRNA-155/(d) in the resulting duplexes are fully base-paired with the miRNA strands. (The miRNAs include single-strand tethers at their 3′-ends.) In the presence of Exo III, the 3′-ends of the duplexes miRNA-21/(c) and of the miRNA-155/(d) are digested, leading to the release of miRNA-21 or of miRNA-155 from the duplexes, thus allowing the participation of the miRNA-21 or of miRNA-155 in additional unlocking cycles (amplification of the release processes). FIG. 6(A), curve (i), shows the time-dependent fluorescence of DOX released from the DOX-loaded T_{A}/NMOFs, in the presence of miRNA-21, 200 nM, and Exo III, 1 U/μL. For comparison, the release of DOX from the same NMOFs in the absence of Exo III is shown in curve (ii), FIG. 6(A). The rate of DOX release in the presence of Exo III is 2.5-fold higher as compared to the DOX release in the absence of Exo III, which is consistent with the improved unlocking of the carriers by the Exo III-stimulated regeneration of the miRNA-21. FIG. 6(B), curve (i) shows the fluorescence values of the released DOX from the DOX-loaded T_{A}/NMOFs in the presence of variable concentrations of miRNA-21 and a constant concentration of Exo III, 1 U/μL, after a fixed time interval of 90 minutes. For comparison, the release of DOX from the DOX-loaded T_{A}/NMOFs under similar conditions, yet in the absence of Exo III, are shown in curve (ii). Clearly, the release of DOX from the carriers are significantly enhanced in the presence of Exo III, due to the biocatalytic regeneration of miRNA-21. FIG. 6(C), curve (i), depicts the time-dependent release of CPT from the CPT-loaded T_{B}/NMOFs upon treatment of the carriers with miRNA-155 of 200 nM, in the presence of Exo III, 1 U/μL, as the miRNA regeneration biocatalyst. For comparison, the release of CPT from the carriers, in the absence of Exo III, under similar conditions is shown in curve (ii). Subjection of the carriers to Exo III results in a 2-fold enhancement in the release of the drugs.

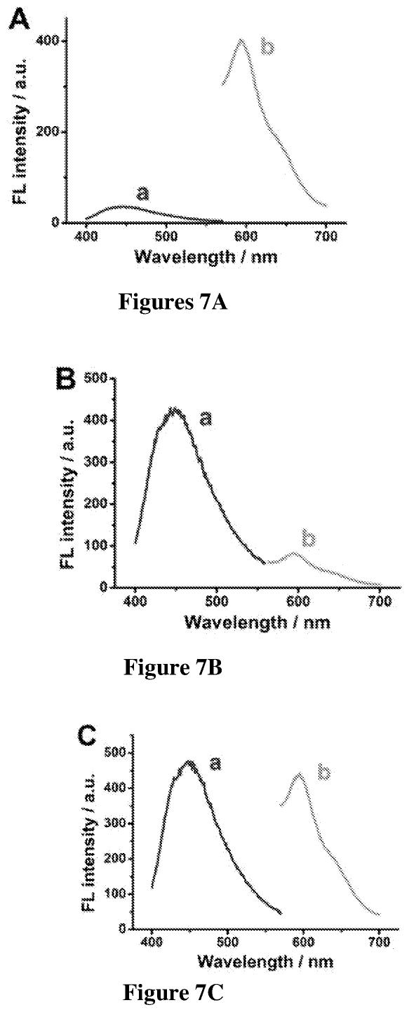

In the next step, the programmed selective release of the drugs from the carriers, in the presence of Exo III was examined, FIG. 7. In this experiment, a mixture of miRNA-21 responsive DOX-loaded T_{A}/NMOFs and miRNA-155 responsive CPT-loaded T_{B}/NMOFs was subjected to Exo III in the presence of either miRNA-21 or miRNA-155 or to both miRNAs. FIG. 7, Panel A, shows the fluorescence spectra of the released drugs from the mixture of NMOFs carriers upon treatment with miRNA-21 200 nM in the presence of Exo III, 1 U·μL⁻¹ (time interval of 90 mins). Only DOX (λₑₘ=590 nm) is being released (curve b), and ineffective release of CPT is observed (curve a). Panel B shows the fluorescence spectra of the released drugs upon treating the mixture of NMOFs with miRNA-155, in the presence of Exo III. Only CPT (λₑₘ=430 nm) is being released (curve a), and inefficient release of DOX is observed (curve b), demonstrating the selective programmed miRNA-dictated release of the drugs. Panel C shows the fluorescence spectra of DOX (curve b) and CPT (curve a) that are released from the mixture of NMOFs, in the presence of added miRNA-21 and miRNA-155.

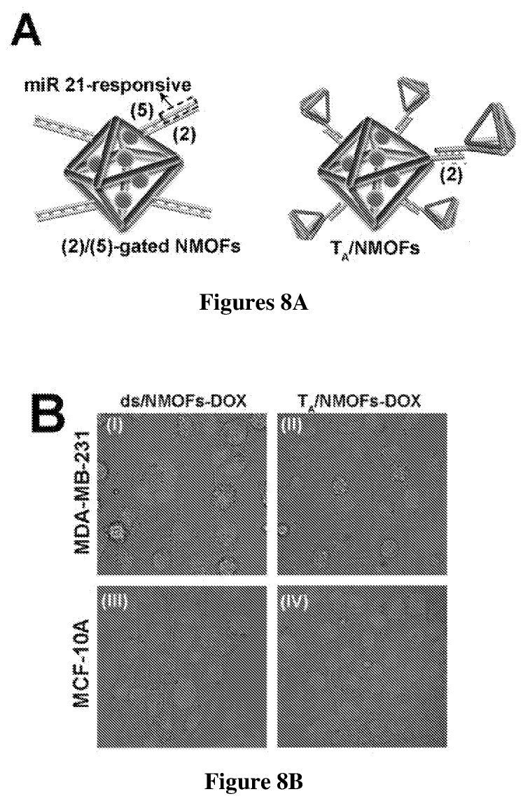

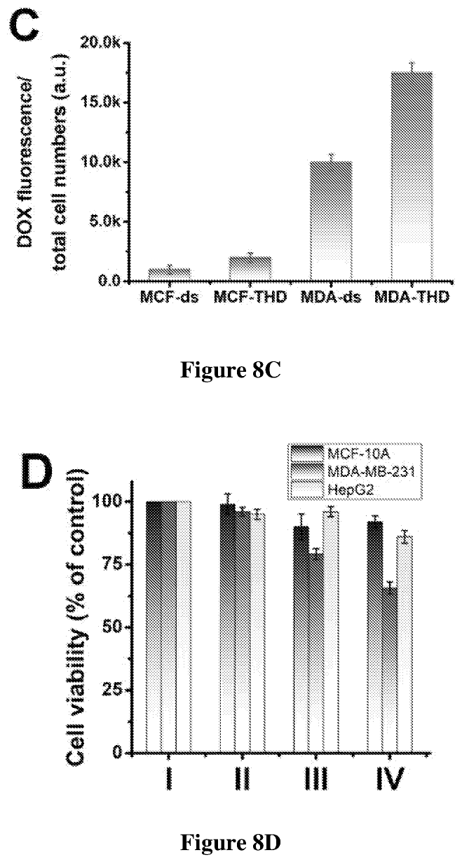

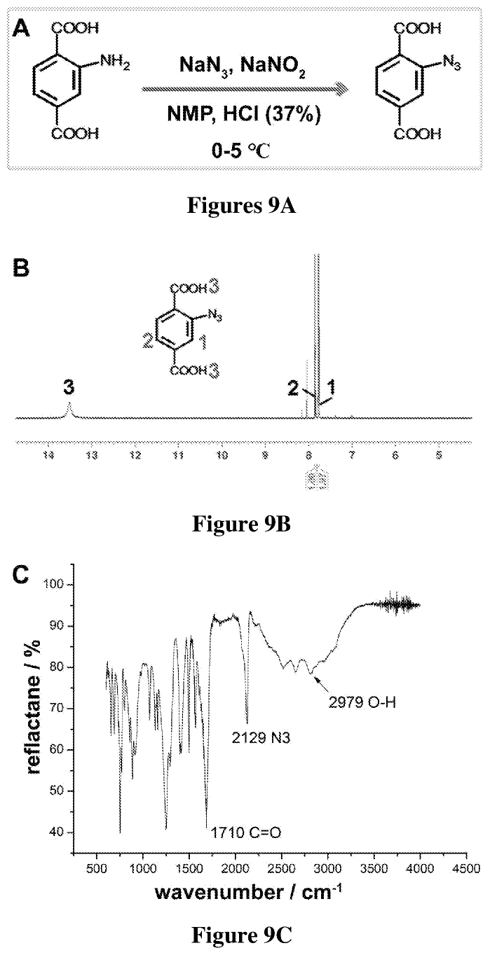

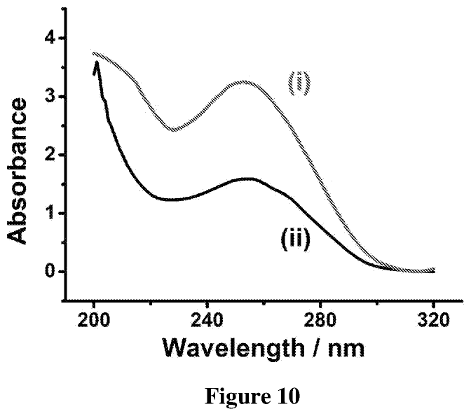

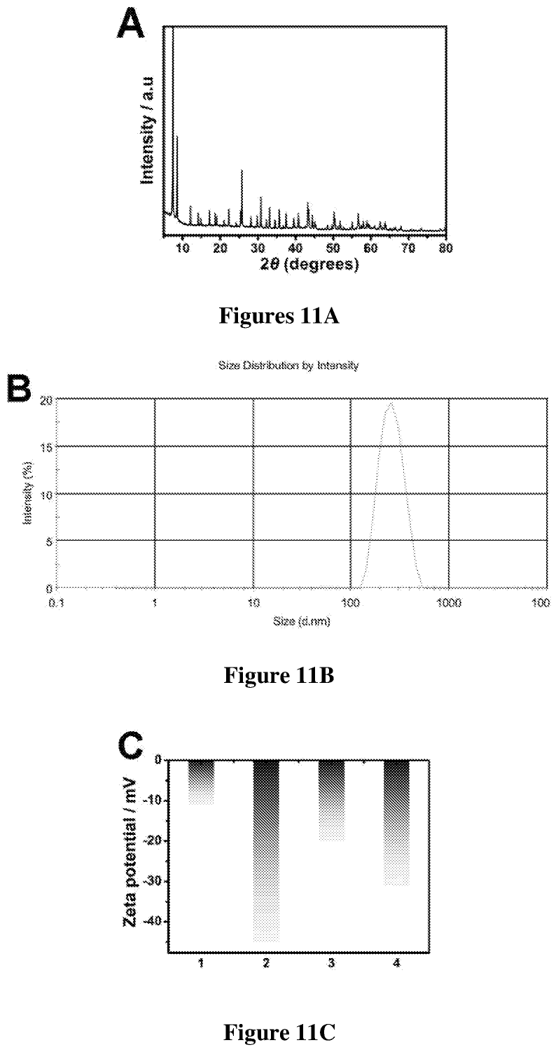

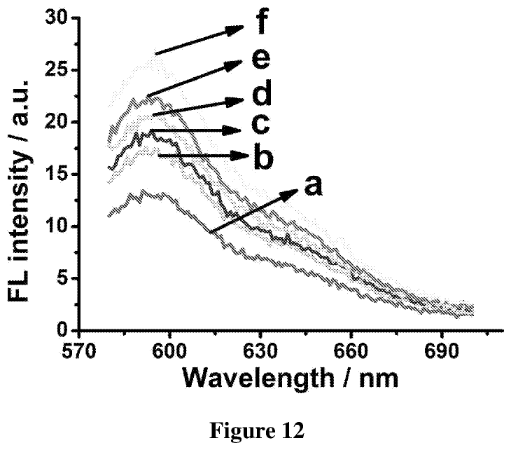

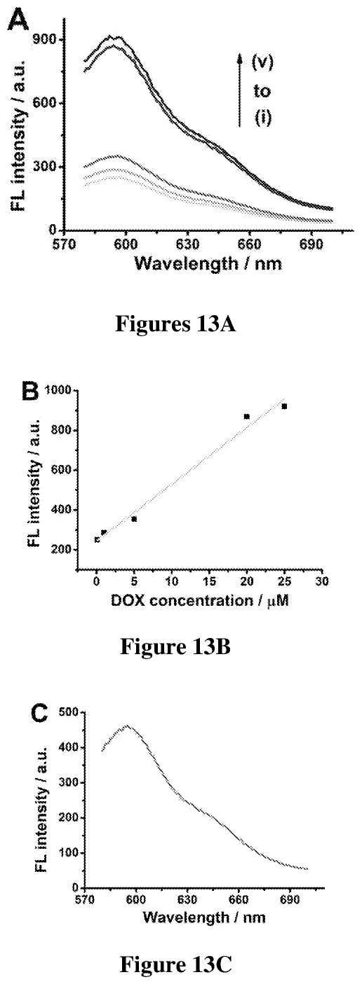

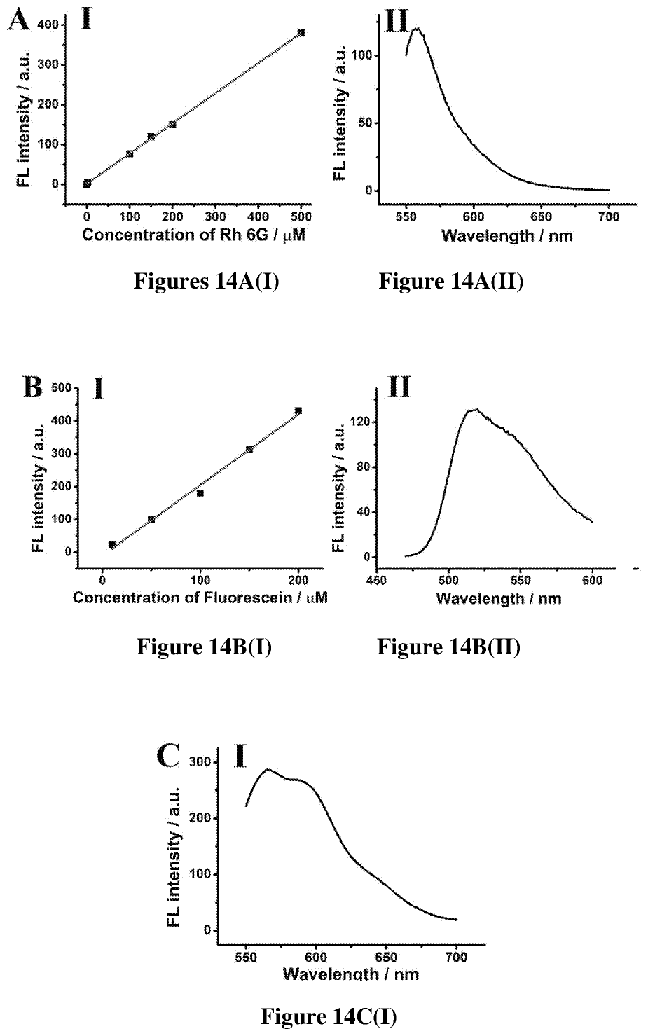

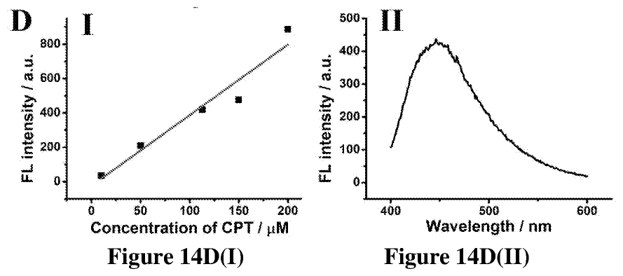

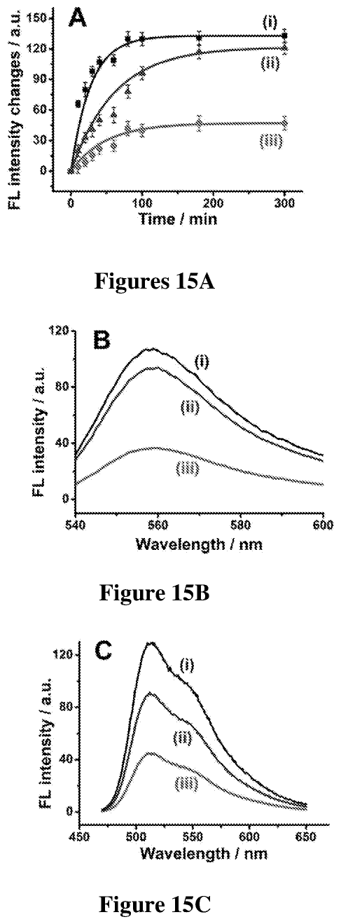

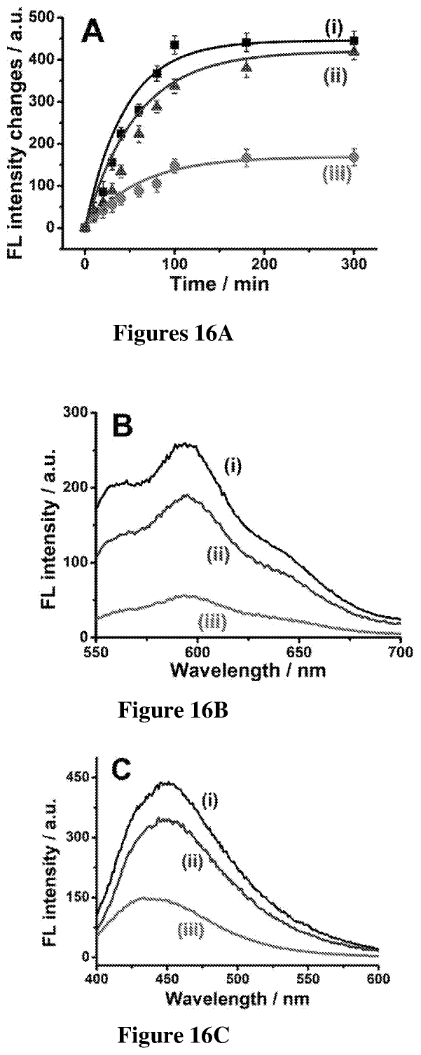

The results demonstrate the sense-and-treat capacities of the miRNA-responsive DNA-tetrahedra-gated drug-loaded NMOFs. In the next step, the cell permeation capacity and cytotoxicity of the DOX-loaded T_{A}/NMOFs towards the cancer cells was compared to a control system consisting of DOX-loaded NMOFs gated by a duplex (2)/(5) miRNA-21 stimuli-responsive locks. In both DNA-tetrahedra and duplex gates, the locking units were engineered to allow the Exo III-stimulated miRNA regeneration process. The structures of (2)/(5)-gated NMOFs and T_{A}/NMOFs are shown in FIG. 8(A). Towards these studies, the cytotoxicity of DOX-loaded miRNA-21 responsive T_{A}/NMOFs towards MDA-MB-231 breast cancer cells, in comparison to non-malignant MCF-10A breast epithelial cells and HepG2 liver cancer cells was examined. It was found that the permeation of the drug-loaded miRNA-21 responsive T_{A}/NMOFs into the MDA-MB-231 cancer cells is ca. 1.75-fold enhanced as compared to the analog (2)/(5) duplex-gated NMOFs (ds/NMOFs), FIG. 8(B), Panel I and Panel II, suggesting that the tetrahedra modules, indeed, improve the permeation capacity of NMOFs into the MDA-MB-231 cells. In addition, the permeation of the DOX-loaded miRNA-21 responsive T_{A}/NMOFs into the MDA-MB-231 cells is 7-fold higher than the permeation into the MCF-10A epithelial cells, FIG. 8(B), Panels II and IV. The permeation of the drug-loaded miRNA-21-responsive T_{A}/NMOFs into the MCF-10A epithelial cells is ca. 2-fold enhanced as compared to the analog (2)/(5) duplex-gated NMOFs, FIG. 8(B), Panel III and Panel IV. The integrated fluorescence intensities of the respective systems, in the form of a “bar” presentation, are shown in FIG. 8(C). FIG. 8(D) shows the respective cell viabilities, in the form of a “bar” presentation, upon treatment of the cells with an identical amount of DOX-loaded NMOFs (interaction time-interval of 3 days). For comparison, the cell viability of non-treated cells is shown in FIG. 8(D), panel I. The results in panel II show that the non-loaded miRNA-21-responsive T_{A}/NMOFs have no effect on the viability of the MDA-MB-231 cells, MCF-10A cells and HepG2 cells. The non-malignant MCF-10A breast epithelial cells are almost unaffected by the DOX-loaded miRNA-21 responsive T_{A}/NMOFs and (2)/(5) duplex-gated NMOFs, consistent to the lack of miRNA-21 in the MCF-10A breast epithelial cells, panel III and panel IV, FIG. 8(D). The cytotoxicity of the DOX-loaded miRNA-21 responsive T_{A}/NMOFs toward the MDA-MB-231 cells reveals after three days a 35% cell death while the (2)/(5) duplex-gated DOX-loaded NMOFs show only ca. 20% cell death. These results are consistent with the improved permeation of the tetrahedra-functionalized drug-loaded nanocarriers into the MDA-MB-231 cells. In addition, a less cytotoxic effect of the DOX-loaded miRNA-21-responsive T_{A}/NMOFs on the HepG2 liver cancer cells is observed, consistent with the lack of miRNA-21 in the cancer cells, panel IV, FIG. 8(D).

The present invention thus provides functional metal organic framework nanoparticles (NMOFs) as “sense-and-treat” drug carriers. These include DNA-tetrahedra modified drug-loaded NMOFs. The DNA tetrahedra nanostructures associated with the NMOFs provide several unique features that “upgrade” the drug carrying capacities and nanomedical applications of the NMOFs. These include the enhanced cell permeation of the NMOFs carriers, the possibility to functionalize the tetrahedra nanostructures with aptamer units acting as cell targeting units that enhance the permeation and specificity of the carriers, and most importantly, the possibility to build on the DNA tetrahedra modules stimuli-responsive locks for the dictated gating and unlocking the carriers and for the integration of amplification machineries to release the loaded drugs. The study has introduced two kinds of stimuli-responsive locks to get the carriers: One included pH-responsive, i-motif, gates that are being unlocked by the slightly acidic conditions present in cancer cell environments. The second unlocking mechanism involved miRNAs-responsive gates being unlocked in the presence of miRNAs specific to cancer cells (miRNA-21 present in MDA-MB-23 breast cancer cells and miRNA-155, a specific biomarker for HepG2 liver cancer cells). The two kinds of stimuli-responsive DNA-tetrahedra gated NMOFs revealed sense-and-treat functionalities by responding to the acidic microenvironments or to cancer-specific biomarkers in triggering the release of the drugs and retaining the benefits of DNA-tetrahedra nanostructures. In addition, the versatility in tailoring different miRNAs gates allowed the miRNA-guided multiplexed release of different drugs from a mixture of gated carriers. Beyond the significance of the study introducing functional drug carriers for personalized medicine, the importance of the study rests on the versatility of the principles and the broad future applications that can be envisaged. For example, the functionalization of other carriers with the DNA tetrahedra is anticipated to allow the development of other superior carriers for drug release or imaging applications. In addition, the information encoded in the base sequences of DNA is anticipated to allow the engineering of other switchable and triggered locks of enhanced biomedical treatment efficacy. For example, the design of aptamer-gated drug-loaded DNA-tetrahedra NMOFs could lead to the release of the drugs through the formation of aptamer-ligand complexes. This would allow cooperative therapeutic sense-and-treat biomedical applications through the release of drugs and simultaneously use the therapeutic functions of aptamer-ligand complexes.

### EXPERIMENTAL SECTION

Materials and Instruments. 2-[4-(2-Hydroxyethyl)piperazin-1-yl]ethanesulfonic acid sodium salt (HEPES), sodium chloride, magnesium chloride, doxorubicin hydrochloride (DOX), camptothecin (CPT), fluorescein, Rhodamine 6G, N, N, N′, N′-tetramethy-lethylenediamine (TEMED), acrylamide solution (40%), agarose, ammonium persulphate, dimethylformamide (DMF), dibenzocyclooctyne-sulfo-N-hydroxysuccinimidyl ester (DBCO-sulfo-NHS), sodium azide (NaN₃), sodium nitrite (NaNO₂), HCl (37%), N-methyl-2-pyrrolidone (NMP), zirconium oxychloride (ZrOCl₂) and 2-aminoterephthalic acid were bought from Sigma-Aldrich. DNA oligonucleotides were synthesized and purified at Integrated DNA Technologies Inc. (Coralville, IA). SYBR Gold nucleic acid gel stain was purchased from Invitrogen. Ultrapure water from NANOpure Diamond (Barnstead) source was applied throughout the whole experiments.

A Magellan XHR 400L scanning electron microscope (SEM) was employed to characterize the microcarriers. Fluorescence spectra was measured with a Cary Eclipse Fluorometer (Varian Inc.). The excitation of DOX, CPT, Rhodamine 6G and fluorescein were excited at 480, 423 nm, 420 nm and 480 nm. The emission of DOX, CPT, Rhodamine 6G and fluorescein were measured at 590, 450 nm, 560 nm and 520 nm. The concentrations of DNA oligonucleotides were monitored using a UV-2401PC (SHIMADAZU) spectrophotometer. The gel experiment was run on a Hoefer SE 600 electrophoresis unit.

The sequences of all nucleic acids used in this application are composed of: (from 5′ to 3′) in parenthesis showing the SEQID number in the sequence listing.

The pH responsive tetrahedra:

S1 (SEQID1):
ACACTACGTCAGAACAGCTTGCATCACTGGTCACCAGAGTACCCTAAC
CCTAACCCTAACCCTACT
S2 (SEQID2):
GGTCCTACGAGCGAGTTGATGTGATGCAAGCTGAATGCGAG
S3 (SEQID3):
GGTGGTGGTGGTTGTGGTGGTGGTGGTTTTCTGACGTAGTGTATGCAC
AGTGTAGTTAGGACCCTCGCAT
S4 (SEQID4):
TCAACTCGCTCGTTACTACACTGTGCAATACTCTGGTGACC
(SEQID5) Anchor (1):
NH₂-(CH₂)₆-AGTAGGGTTAGG
(SEQID6) Strand (2):
GGTGGTGGTGGTTGTGGTGGTGGTGGCCCTAACCCTAACCCTAACCCT
ACT

The miRNA-21 responsive tetrahedra (TA):

S1-21/(5) (SEQID7):
ACACTACGTCAGAACAGCTTGCATCACTGGTCACCAGAGTAAAAAAA
ACATCAGTCTGATAAGCTA
S1-21-no Exo III responsive (SEQID8):
ACACTACGTCAGAACAGCTTGCATCACTGGTCACCAGAGTAAAATCAA
CATCAGTCTGATAAGCTAAAAAA
S2-21 (SEQID9):
GGTCCTACGAGCGAGTTGATGTGATGCAAGCTGAATGCGAG
S3-21 (SEQID10):
TCTGACGTAGTGTATGCACAGTGTAGTTAGGACCCTCGCAT
S4-21 (SEQID11):
TCAACTCGCTCGTTACTACACTGTGCAATACTCTGGTGACC

The miRNA-155 responsive tetrahedra (TB):

S1-155 (SEQID12):
ACACTACGTCAGAACAGCTTGCATCACTGGTCACCAGAGTAAAAAAA
CCTATCACGATTAGCATTAA
S1-155-no Exo III responsive (SEQID13):
ACACTACGTCAGAACAGCTTGCATCACTGGTCACCAGAGTAAAATCAA
CATCAGTCTGATAAGCTAAAAAA
S2-155 (SEQID14):
GGTCCTACGAGCGAGTTGATGTGATGCAAGCTGAATGCGAG
S3-155 (SEQID15):
TCTGACGTAGTGTATGCACAGTGTAGTTAGGACCCTCGCAT
S4-155 (SEQID16):
TCAACTCGCTCGTTACTACACTGTGCAATACTCTGGTGACC

Generation of DNA tetrahedra. The DNA tetrahedra depicted in FIG. 1(C), consisted of four sequences, were prepared as follows. A mixture of (S1), (S2), (S3) and (S4) (2 μM each) in HEPES buffer (10 mM, 20 mM MgCl2, pH=7.2) was annealed at 95C for 5 min, subsequently, cooled down to 4 C, and allowed to equilibrate at 25 C for 2 hours, yielding DNA tetrahedra. The tetrahedra A and tetrahedra B were synthesized by following the same procedure.

Synthesis of NMOFs. The preparation of NMOFs was according to the reported method (Morris, W.; Briley, W. E.; Auyeung, E.; Cabezas, M. D.; Mirkin, C. A. Nucleic Acid-Metal Organic Framework (MOF) Nanoparticle Conjugates. J. Am. Chem. Soc. 2014, 136, 7261-7264). First, 50 mg of 2-azido-terephthalic acid and 21 mg of ZrOCl2 were mixed together in DMF (4 mL). Then, 2 mL of acetic acid were added to the mixture that was heated in an oven at 90° C. for 18 hours. After that, the resulting NMOFs were centrifuged and washed with DMF, triethylamine/ethanol (1:20, V/V), and ethanol.

Preparation of DBCO-functionalized DNA (1) or (3) or (4). To link the DBCO functional groups to nucleic acid (1) or (3) or (4) (DBCO=dibenzocyclooctyne), 200 μL of 1×10-4 M (1) or (3) or (4) was added to 20 μL of 5×10-2 M DBCO-sulfo-NHS (dissolved in water) and shaked overnight. The obtained solution was filtered through a MicroSpin G-25 columns (GE-Healthcare) to produce the pure DBCO-DNA.

Synthesis of DNA (1)-, (3)-, (4)-functionalized NMOFs. Initially, NMOF nanoparticles (1 mg, 1 mL) were treated with DBCO-DNA (1) or (3) or (4) (100 nmol). Then, the resulting solution was diluted with NaCl to a final concentration of 0.5 M and incubated over 6 h. Subsequently, the resulted solution was shaked at 40° C. for 72 h. The DNA (1) or (3) or (4)-functionalized NMOFs were washed in water to remove unbound nucleic acids.

The drug/dye loading of NMOFs. To load the drugs, 1 mg of DNA (1)-, (3)-, (4)-functionalized NMOFs were shaked with doxorubicin, DOX (1 mL, 14.5 mM), camptothecin, CPT (1 mL, 0.193 mM), Rhodamine 6G (1 mL, 0.216 mM) or fluorescein (1 mL, 0.12 mM) for 12 h in 1 mL water. The DNA (1)-, (3)-, (4)-functionalized NMOFs were then transferred to a buffer solution and hybridized with the nucleic acid (2), the pH-responsive tetrahedra, the tetrahedra A or the tetrahedra B, respectively, resulting in the locked state of the NMOFs encapsulated the respective drugs/dyes. 12 hours later, the NMOFs were washed several times to remove the unloaded drugs. The drugs/dyes-loaded tetrahedra-gated or duplex-gated NMOFs were kept at 4° C. for further use.

pH-responsive unlocking of the tetrahedra A-gated NMOFs and the release of the DOX. The pH-responsive DOX-loaded NMOFs, 0.1 mg, were subjected to 1mL buffer solutions, at pH 7.4 or pH 5.0. At appropriate time intervals, samples of the mixture are centrifuged to precipitate the NMOFs (10 000 rpm for 2 minutes). The fluorescence of the released loads in the supernatant solution was measured using a Cary Eclipse Fluorescence Spectro- photometer (Varian Inc.).

miRNA-triggered unlocking of the NMOFs and the release of the encapsulated dyes/drugs. The tetrahedra A or tetrahedra B-gated, drug model—(Rhodamine 6G or fluorescein) or drug (DOX or CPT)-loaded NMOFs, at a concentration corresponding to 0.1 mg/mL, 1 mL, were subjected to the respective miRNAs (miRNA-21 or miRNA-155) with different concentrations to unlock the NMOFs and release the dyes/drugs. The NMOF solutions were treated with different concentrations of miRNAs and a fixed concentration of Exo III (1 U/μL). At a certain time-intervals, the respective sample solutions were centrifuged to precipitate the NMOFs (10 000 rpm for 2 minutes), and the fluorescence of the released loads in the supernatant solutions was measured using a Cary Eclipse Fluorescence Spectrophotometer (Varian Inc.).

Cell culture. Normal breast cells (MCF-10A) were maintained in complete growth medium consisting of 1:1 mixture of Dulbecco's modified Eagle's medium and Ham's F12 medium supplemented with horse serum (5%), epidermal growth factor (20 ng/mL), cholera toxin (CT, 0.1 μg/mg), insulin (10 μg/mL), hydrocortisone (500 ng/ml), and penicillin/streptomycin (1 unit/mL). Human breast cancer cells (MDA-MB-231) were grown in 5% CO2 RPMI-1640 medium supplemented with 10% FCS, L-glutamine, and antibiotics (Biological Industries). Cells were plated one day prior to the experiment on 96-well plates for cell viability or on u-slide 4 well glass bottom (ibidi) for confocal microscopy.

Cell viability experiments. Cell viability was assayed after incubation of the DNA duplex-gated NMOFs or DNA tetrahedra-gated NMOFs loaded with DOX in MCF-10A, MDA-MB-231 cells and HepG2 cells planted at a density of 1.2×10⁴ cells per well in 96-well plates. After 6 hours incubation with the NMOFs, 60 μg/mL, cells were washed intensively with growth medium and the cell viability was determined with the fluorescent redox probe, Presto-Blue. The fluorescence of Presto-Blue was recorded on a plate-reader (Tecan Safire) after 1 h of incubation at 37° C. (λₑₓ=560 nm; λₑₘ=590 nm).

Confocal microscopy measurements. Cells were planted in μ-slide 4 well glass bottom on one day prior to the experiment. Cells were incubated with the DOX-loaded DNA duplex-gated NMOFs or DNA tetrahedra-gated NMOFs, 60 μg/mL, for 6 hours and then washed with DMEM-Hepes twice. DOX fluorescence in cells was monitored with the confocal microscopy (the Olympus FV3000 confocal laser-scanning microscope) and all images were analyzed with Image J software.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A nanoparticle loaded with at least one active agent; wherein said at least one active agent is being locked within said particle by at least one DNA-nanostructure attached to said particle via at least one stimuli-responsive nucleic acid-based bridging unit.

2. A nanoparticle according to claim 1, wherein said DNA-nanostructure is selected from a four-way junction DNA, DNA-tile, DNA-crystal, DNA-cage, DNA-brick, DNA-polygonal structure, DNA-wireframe, DNA-tweezers, DNA-amphiphile, tetrahedra DNA, and any combination thereof.

3. A nanoparticle according to claim 1, wherein said at least one stimuli-responsive nucleic acid-based bridging unit comprises at least one stimuli-responsive sequence selected from a hairpin strand, a pH-responsive strands, a DNAzyme, a G-quadruplex, a stem-loop strand, an aptamer, a pathogen-specific sequence, microRNA, catalytically degradable sequence and any combinations thereof.

4. A nanoparticle according to claim 1, wherein said at least one stimuli-responsive nucleic acid-based bridging unit further comprises a complementary sequence.

5. A nanoparticle according to claim 1, wherein said at least one DNA-nanostructure further comprises at least one targeting nucleic acid-based unit.

6. A nanoparticle according to claim 1, wherein said at least one DNA-nanostructure further comprises at least one targeting nucleic acid-based unit selected from a hairpin strand, a pH-responsive strands, a DNAzyme, a G-quadruplex, a stem-loop strand, an aptamer, a pathogen-specific sequence, microRNA, catalytically degradable sequence and any combinations thereof.

7. A nanoparticle according to claim 1, wherein said stimuli is selected from pH, radiation, light, temperature, gas, salt, metal, chemical reducing agent, carbohydrate, enzyme, magnetic field, ultrasonic or microwave agitation and any combinations thereof.

8. A nanoparticle according to claim 1, wherein said at least one active agent is selected from a drug, a pro-drug, a labeling agent, a hormone, a reactive compound capable of forming at least one stimulus, a nucleic acid sequence, a steroid, a sensor, a transistor, a radioactive agent and any combinations thereof.

9. A composition comprising at least one nanoparticle according to claim 1.

10. (canceled)

11. A process for the preparation of a nanoparticle loaded with at least one active agent; wherein said at least one active agent is being locked within said particle by at least one DNA-nanostructure attached to said particle via at least one stimuli-responsive nucleic acid-based bridging unit; said process comprising the steps of:
a. attaching at least one first nucleic acid sequence to a nanoparticle; thereby providing a modified nanoparticle;
b. loading said modified nanoparticle with at least one active agent; thereby providing a loaded modified nanoparticle;
c. hybridizing said loaded modified nanoparticle with at least one DNA-nanostructure comprising at least one second nucleic acid sequence; wherein at least one of said first and second nucleic acid sequence is a stimuli-responsive sequence; thereby forming said at least one stimuli-responsive nucleic acid-based bridging unit attaching said at least one DNA-nanostructure with said nanoparticle and locking said at least one active agent within said particle.

12. A process according to claim 11, wherein said first nucleic acid sequence is a stimuli-responsive sequence.

13. A process according to claim 11, wherein said second nucleic acid sequence is a stimuli-responsive sequence.

14. A process according to claim 11, wherein said at least one DNA-nanostructure further comprises at least one targeting nucleic acid-based unit.

15. (canceled)
